(19) United States
(12) Patent Application Publication
(10) Pub. No.: US 2008/0290045 A1
(43) Pub. Date: Nov. 27, 2008

(54) Process and apparatus for water decontamination

(76) Inventors: B. Keel Robinson, Richmond, CA (US); Douglas C. Gustafson, Antioch, CA (US)

Correspondence Address: King & Spalding LLP, P.O. Box 889, Belmont, CA, 94002-0889, US

(21) Appl. No.: 12/148,684

(22) Filed: Apr. 21, 2008

Related U.S. Application Data
(60) Provisional application No. 60/925,645, filed on Apr. 19, 2007.
(60) Provisional application No. 60/995,834, filed on Sep. 28, 2007.

(51) Int. Cl. C02F 1/32; C02F 1/78; A61L 2/10

(52) U.S. Cl. 210/748; 422/24

(57) ABSTRACT

Apparatus and method relating to water decontamination, and more particularly, to removing NDMA and other organic contaminants from water using ultraviolet light in combination with ozone or ozone and peroxide, are described.

### PRIORITY

The present application claims priority to U.S. Provisional Application Ser. Nos. 60/925,645, filed on Apr. 19, 2007, 60/995,834, filed on Sep. 30, 2007, and Attorney Docket No. 64524-8024 (entitled, “Modular Sidestream Apparatus for Water Treatment”), filed on Apr. 3, 2008, which are hereby incorporated by reference in their entirety.

### TECHNICAL FIELD

The present apparatus and method relate to water decontamination, and more particularly, to removing NDMA and other organic contaminants from water using ultraviolet light in combination with ozone or ozone and peroxide.

### BACKGROUND

N-nitrosodimethylamine (NDMA) is the best known member of the N-nitrosomines, a class of organic compounds that are carcinogenic, mutagenic, and teratogenic. Exogenous sources of NDMA in water include rocket fuels and there components, cutting oils, tobacco smoke, herbicides, pesticides, aminopyrine in drug formulations, and rubber products. NDMA is also formed as a disinfection by-product of chlorine/chloramine treatment of water, particularly water having elevated levels of organic nitrogen and bromide. There is direct correlation between the levels of chlorination/chloramination, which is typically performed at 1-5 mg/L, and the amount of NDMA formed, typically 20-100 ng/L.

The U.S. Environmental Protection Agency (EPA) has adopted an NDMA clean-up standard of 0.7 ng/L and the State of California (USA) has set a maximum permissible level of NDMA in drinking water of 10 ng/L. Federal and State environmental hazard assessments recommend a level of only 2 ng/L, although there is no legislation to that effect. The minimum level of detection for NDMA is typically in the range of 0.5-2 ng/L.

While volatile, NDMA is highly soluble in water, and removal by volatilization, filtration, and reverse osmosis is ineffective. However, UV light in the 225-250 nm wavelength range causes photolysis of NDMA to dimethylamine (DMA) and nitrite, as well as nitrate, formaldehyde, and formate, by breaking the N—N bond. While UV treatment destroys NDMA, it suffers from at least two major drawbacks. First, UV destruction of NDMA requires much more energy (about 10-25 times) than is required for disinfection. This makes NDMA removal an expensive and environmentally unfriendly proposition. Second, the “daughter” products of UV photolysis can reform NDMA, particularly in the presence of chlorine or chloramine, which is often added as a residual disinfectant.

To reduce the reformation of NDMA, UV treatment is typically accompanied by hydrogen peroxide treatment to oxidize the daughter products; thereby preventing reformation. This requirement for hydrogen peroxide further increases the cost of the process. Moreover, UV treatment is not very effective for removing 1,4-dioxane and volatile organic compounds (VOCs), requiring even additional amounts of UV energy and peroxide.

An effective and economical decontamination process for oxidizing such contaminants as NDMA, 1,4-dioxane, and VOCs is needed.

### SUMMARY

The following aspects and embodiments thereof described and illustrated below are meant to be exemplary and illustrative, not limiting in scope.

In one aspect, a method for removing N-nitroso-dimethylamine (NDMA) and NDMA derivatives from water is provided, comprising contacting the water with UV light, and then contacting the water with ozone, thereby reducing the amount of NDMA and NDMA derivatives.

In some embodiments, the NDMA derivative is dimethylamine (DMA).

In some embodiments, the ozone is provided in the absence of hydrogen peroxide. In other embodiments, the ozone is provided in combination with hydrogen peroxide.

In some embodiments, the water further included volatile organic compounds (VOCs) and the method reduces the amount of VOCs in the water. In some embodiments, the method disinfects the water. In particular embodiments, the disinfecting includes killing viruses and coliforms.

In some embodiments, the method utilizes a modular sidestream injection apparatus.

In a related aspect, a method for removing N-nitroso-dimethylamine (NDMA) and NDMA derivatives from water is provided, comprising contacting the water with ozone, and then contacting the water with UV light, thereby reducing the amount of NDMA and NDMA derivatives.

In some embodiments, the NDMA derivative is dimethylamine (DMA). In some embodiments, the water further included volatile organic compounds (VOCs) and the method reduces the amount of VOCs in the water.

In some embodiments, the ozone is provided in the absence of hydrogen peroxide. In other embodiments, the ozone is provided in combination with hydrogen peroxide.

In some embodiments, the method disinfects the water. In particular embodiments, the disinfecting includes killing viruses and coliforms.

In some embodiments, the method further includes contacting the water with ozone after contacting the water with UV light.

In some embodiments, the UV light causes the formation of hydroxyl radicals from ozone.

In some embodiments, treatment of the water with ozone increases the UV transmittance of the water prior to treating the water with UV light.

These and other objects and features of the invention are made more fully apparent in the following detailed description of the invention.

### BRIEF DESCRIPTION OF THE DRAWINGS

FIGS. 1A-1H illustrate different operating configurations for the present apparatus and methods.

FIG. 2A-2D illustrate further operating configurations for the present apparatus and methods.

FIG. 3 shows a schematic of a modular sidestream ozone/advanced oxidation apparatus.

FIGS. 4A and 4B show a side view of a main reactor or sidestream of an exemplary ozone/advanced oxidation apparatus. FIG. 4C shows an exemplary ozone/advanced oxidation apparatus including a main reactor and two sidestreams.

FIGS. 5A-5E show side views (FIGS. 5A and 5B) and cross-section views (FIGS. 5C-5E) of injector modules having different injector configurations. FIG. 5F shows a side view of an exemplary injector.

FIGS. 6A-6D show side views (FIGS. 6A and 6C) and a cross-section view (FIG. 6D) of mixer modules having different mixer configurations.

FIG. 7 shows a side view of an exemplary modular main reactor apparatus for receiving ozone and hydrogen peroxide via a sidestream.

### DETAILED DESCRIPTION

### I. Overview of the Apparatus and Method

The present apparatus and method relate to the use of UV energy for photolytic degradation of organic contaminants and disinfection, in combination with ozone and, optionally, hydrogen peroxide. As used herein, combined ozone/hydrogen peroxide treatment is referred to as advanced oxidation, which encompasses HIPOX™ apparatus and methods, which are used by Applied Process Technology, Inc. (Pleasant Hill, Calif., USA). Note that while HIPOX™ apparatus and methods may involve elevated operating head pressures, the present apparatus and methods are not limited to a particular range of operating pressures.

While many organic contaminants can be removed using the present apparatus and methods, a contaminant of particular interest is N-nitrosodimethylamine (NDMA). Conventional UV apparatus methods can be effective at removing NDMA from water via photolysis but requires the presence of hydrogen peroxide to oxidize the products of photolysis (i.e., NDMA “daughter” products) to prevent NDMA reformation. UV photolysis of NDMA already requires much more energy than required for UV disinfection, and the need to add hydrogen peroxide further increases the cost. In addition, UV photolysis of contaminants such as 1,4-dioxane and various volatile organic compounds (VOCs) is inefficient and requires even additional UV energy and hydrogen peroxide. Thus, while UV treatment remains a preferred method for performing water disinfection, it leaves much to be desired for removing organic contaminants.

The present apparatus and method are based on the idea of combining UV photolysis for degradation of NDMA with the use of ozone, alone or in an advanced oxidation configuration, to destroy NDMA daughter products. Moreover, since the destruction of 1,4-dioxane, endocrine disrupting compounds (EDCs), 1,2,3,-trichloropropane, trichloroethylene (TCE), and other VOCs is driven by reactive oxygen species, rather than photolysis, ozone and advanced oxidation are more effective than UV energy in removing these compounds. In addition, while UV energy is efficient in disinfecting water of coliforms, ozone and advanced oxidation assist in removing viruses.

The present apparatus and method offer several advantages over conventional water treatment apparatus and method. First, UV treatment for photolytic degradation of NDMA need not require the addition of hydrogen peroxide since treatment with ozone effectively destroys daughter products, such as dimethylamine (DMA). The combined operation and maintenance cost of using UV energy and ozone (or even advanced oxidation) are expected to be lower than for UV energy and hydrogen peroxide for removing contaminants such as NDMA. Second, the combination of UV energy and ozone (or advanced oxidation) efficiently removes NDMA and its derivatives (as well as other VOCs) and disinfects water more efficiently than UV energy and hydrogen peroxide, providing a powerful water treatment method for decontamination as well as disinfection.

Ozone or advanced oxidation can also be used to clarify water prior to UV treatment, or used simultaneous with UV treatment to clarify water, thereby increasing UV transmittance to increase the efficiency of UV treatment and lower UV energy costs. UV energy also promotes the formation of hydroxyl radicals from ozone, thereby making the ozonation or advanced oxidation process more efficient.

### II. Definitions

Prior to describing aspects and embodiments of the present apparatus and method, the following terms are defined for clarity. Terms and abbreviations not defined should be accorded their ordinary meaning as used in the art. Note also that singular articles, such as “a” and “an” encompass the plural, unless otherwise specified or apparent from context.

As used herein, a “substantially undetectable” level of a particular contaminant refers to either (i) a level below which the contaminant cannot be detected using a technology approved for water testing, (ii) a numerical concentration as described herein, or (iii) a maximum level established by a regulatory body. As used herein, an undetectable level of NDMA is a numerical concentration of about 0.5 ng/L or less, and a nearly undetectable level is a numerical concentration of about 0.2 ng/L or less. Similarly, an undetectable level of nitrate is a numerical concentration of less than about 0.2 N-mg/L and an undetectable level of TCE is a numerical concentration about 0.5 μg/L.

As used herein, “treating” ground water refers to decontaminating, or reducing the levels of a contaminant, in the ground water by performing one or more decontamination steps, as described, herein.

As used herein, “drinking water” refers to water suitable for human and/or animal consumption. Where specified or apparent from context, drinking water may comply with local laws and regulation relating to the levels of various contaminants in the water.

As used herein, “contaminants” present in water include organic chemical contaminants, biological contaminants (i.e., organisms), and particulate matter.

As used herein, “organic contaminants” are chemical compounds present in contaminated water that include predominantly carbon, nitrogen, oxygen, and hydrogen atoms and are not organisms. Exemplary organic contaminants are referred to, herein.

As used herein, “disinfection” refers to removing or destroying organisms present in water, including bacteria, fungus, and viruses.

As used herein, “removal of” or “removing” a contaminant from water refers to reducing the concentration or level of a contaminant by at least 80%, at least 85%, at least 90%, and even at least 90%, compared to an original or reference concentration or level.

As used herein, the following abbreviations have the following meaning, unless otherwise indicated:
ppm=parts per million
ppb=parts per billion
ppt=parts per trillion
UV=ultraviolet radiation
Adv. Ox.=advanced oxidation
EDC=endocrine disrupting compound
VOC=volatile organic compound
TCE=Trichloroethylene
kW=Kilowatts
kWH=kilowatts hour
gal.=Gallon
GPM=gallons per minute
CFM=cubic feet per minute
lb.=pound
mo.=month
HIPOX™=hi-pressure advanced oxidation
ng/L ng/liter
CCF=hundred cubic feet

### III. Combined UV Energy/Ozone Treatment for Water Decontamination

The present apparatus and method relate to the use of UV energy in combination with ozone (or advanced oxidation) to decontaminate water. Operating configurations and features of the apparatus and method are now to be described.

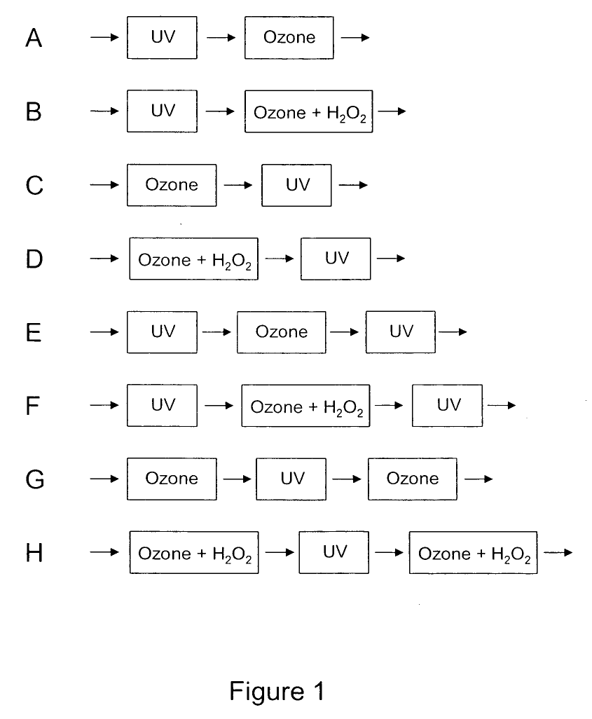

In one operating configuration, influent contaminated water is first treated with UV energy to affect photolysis of NMDA and disinfection, and then treated with ozone to remove NDMA daughter products (FIG. 1A). In a related operating configuration, water is first treated with UV energy and then with advanced oxidation (FIG. 1B).

In another operating configuration, water is first treated with ozone, and then treated with UV energy, wherein residual ozone is present during UV treatment to remove NDMA daughter products produced by UV photolysis (FIG. 1C). In a variation of this operating configuration, water is first treated with advanced oxidation, and then treated with UV energy (FIG. 1D).

In another operating configuration, water is first treated with UV energy, then treated with ozone, and then treated with UV energy again (FIG. 1E). In a variation of this operating configuration, water is first treated with UV energy, then treated with advanced oxidation, and then treated with UV energy again (FIG. 1F).

In another operating configuration, water is first treated with ozone, then treated with UV energy, and then treated with ozone again (FIG. 1G). In a variation of this operating configuration, water is first treated with advanced oxidation, then treated with UV energy, and then treated with advanced oxidation (FIG. 1H).

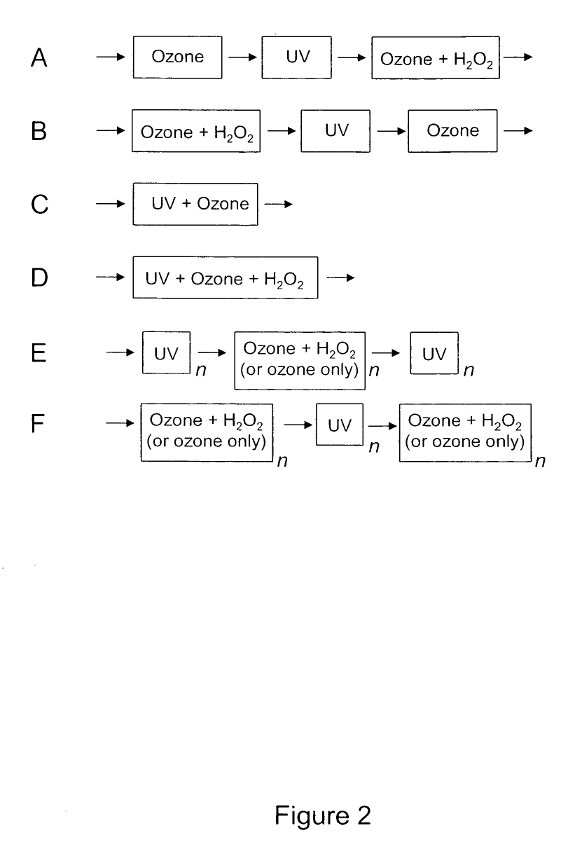

These basic operating configurations can be combined to produce more complex decontamination schemes, e.g., tailored to remove a particular contaminant. For example, water may be treated with ozone, then UV energy, and then advanced oxidation (FIG. 2A), or treated with advanced oxidation, then UV energy, and then ozone (FIG. 2B). UV treatment and ozone treatment (FIG. 2C), or UV treatment and advanced oxidation treatment (FIG. 2D), may also be performed simultaneously, or may be partially overlapping. For example, ozone, optionally with hydrogen peroxide, may be present in water during UV energy treatment. Similarly, UV energy may be applied to water as ozone, optionally with hydrogen peroxide, are added. Note that hydrogen peroxide can be used in combination with UV energy to reduce NDMA reformation, and in combination with ozone in an advanced oxidation process, e.g., to increase the destruction of NDMA and other VOCs. The different uses of hydrogen peroxide will be apparent from the text.

Some operating configurations include additional treatments steps, or repeating a particular treatment step, until a preselected reduction or maximum level of a particular contaminant is achieved. For example, as shown in FIGS. 2E and 2F, each “n” may be independently selected from 1-10, for example, 2, 3, 4, 5, 6, 7, 8, 9, or 10. Other operating configurations avoid repeating the same treatment step, use the fewest total number of treatment steps, minimize the transfer of water from one location to another, and minimize or eliminate the use of hydrogen peroxide.

Certain operating configuration may be preferred under certain situations, depending, e.g., on the contaminants present in the water. In some situations, ozone or advanced oxidation treatment proceeds UV energy treatment to increase the transmittance of water and eliminate 1,4-dioxane and other VOCs prior to using UV energy to remove NDMA, with NDMA daughter products being removed by residual ozone, or hydrogen peroxide, if present. This sequence of treatment step is preferred where the poor transmittance of the untreated water would interfere with UV energy treatment. Alternatively, water is treated first with ozone or advanced oxidation to remove 1,4-dioxane, EDC, 1,2,3,-trichloropropane, TCE, and other VOCs, then with UV to remove NDMA, and then again with ozone or advanced oxidation to remove NDMA derivatives. This sequence of events is preferred when the reformation of NDMA after UV energy treatment is an issue. In other situations, water is first treated with UV and then with ozone or advanced oxidation. The latter order of event may be preferred where poor transmittance of the untreated water is not an issue, and/or where the UV energy treatment is generally effective for water decontamination with the exception of NDMA daughter products, 4-dioxane, EDC, 1,2,3,-trichloropropane, TCE, and other VOCs, which are best removed using ozone or advanced oxidation.

### IV. Types of Contaminants that can be Removed

The present apparatus and method are useful for removing a variety of water-borne contaminants, including N-nitrosomines, a class of potent carcinogens exemplified by N-nitrosodimethylamine (NDMA). NDMA is a carcinogenic, mutagenic, and teratogenic, odorless, yellow, oily liquid, having the empirical formula C₂H₆N₂O and the following structure:

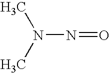

NDMA is formed as a disinfection by-product of chlorine/chloramine treatment of water, particularly water with elevated levels of organic nitrogen and bromide. There is direct correlation between the levels of chlorination/chloramination, which is typically performed at 1-5 mg/L, and the amount of NDMA formed, typically 20-100 ng/L. A number of different reactions produce NDMA, some of which are listed in Table 1. The production of NDMA from nitrite and dimethylamine (DMA) (Table 1, Reaction No. 6), which are both present in food, occurs in the acid conditions of the stomach; therefore endogenous NDMA is produced in the bodies of mammals (including humans) and other animals. Exogenous sources of NDMA include rocket fuels and there components, cutting oils, tobacco smoke, herbicides, pesticides, aminopyrine in drug formulations, and rubber products. Typical levels of NMDA found in various sources are shown in Table 2.

TABLE 1
Examples of chemical reactions that produce NDMA

| No. | Reactions that produce NDMA |
|---|---|
| 1 | Chloramine + DMA → NDMA |
| 2 | Chloramine + UDMH → NDMA |
| 3 | Chloramine + tertiary amines → NDMA |
| 4 | Hypochlorite + DMA → NDMA |
| 5 | Chloramine + chlorinated DMA → NDMA |
| 6 | Nitrite + DMA → NDMA |
| 7 | Chloramine + nitrite + DMA → NDMA |
| 8 | Chloramine + DMA → NDMA |
| 9 | Bromamine + DMA → NDMA |
| 10 | Quaternary amine + chlorine → NDMA |
| 11 | Quaternary amine + chlorine + nitite → NDMA |
| 12 | Quaternary amine + chloramine → NDMA |
| 13 | Quaternary amine + chloramine + nitrite → NDMA |

DMA = dimethylamine;
UDMH = unsymmetrical dimethylhydrazine

TABLE 2
Levels of NDMA found in various sources

| Source | Typical levels | |
|---|---|---|
| Drinking water | Often >10 | ng/L |
| Cured meat | 600-1,000 | ng/kg |
| Fish | 50-6,000 | ng/kg |
| Milk | 90-100 | ng/L |

The U.S. Environmental Protection Agency (EPA) has adopted an NDMA clean-up standard of 0.7 ng/L and the State of California (USA) has set a maximum permissible level of NDMA in drinking water of 10 ng/L. Federal and State environmental hazard assessments recommend a level of only 2 ng/L, although there is no legislation to that effect. The minimum level of detection for NDMA is typically in the range of 0.5-2 ng/L.

The present apparatus and method are particularly useful for maximizing the destruction of NDMA and its derivatives including DMA, other N-nitrosomines, trichloroethylene (TCE), 1,4-dioxane, 1,1-dichloroethene (1,1-DCE), 1,2-dichloroethane (1,2-DCA), 1,2-dichloroethene (1,2-DCE; cis and trans), chloroform (CF), Freon 113, 1,2,3,-trichloropropane, EDCs, tetrachloroethylene (PCE), dichloromethane (DCM), nonylphenol (NP), triclosan (TCS), Bisphenol-A (BPA), estradiol equivalents (EEQ), fluorocarbons, chlorocarbons, chlorofluorocarbons (CFCs), other VOCs, and the like, at minimal costs and with minimal consumption of expensive oxidants (e.g., hydrogen peroxide). Other contaminants that ozone dissolution and advanced oxidation processes are effective in removing include but are not limited to geosmin, 2-methylisoborneol (MIB), mercaptans, 2,3,6-trichloroanisole, iron, manganese, sulfides, chlorine, and MTBE.

The apparatus and methods can also be used to disinfect water of, e.g., coliforms and other bacteria, as well as fungus and viruses. Applying UV energy to contaminated water is a preferred method of removing organisms from water and can be used in combination with ozone and hydrogen peroxide to disinfect and decontaminate water. The increased efficiency and effectiveness of the present apparatus and method should permit implementation of decontamination projects previously considered unfeasible due to the degree of contamination and the expense required.

### V. Studies in Support of the Apparatus and Method

Among the support for the present apparatus and methods are experimental data showing efficacy in removing contaminants present in ground water obtained from several experimental wells, identified as E/F, A, and B. Different amounts of ozone and hydrogen peroxide were used in an advanced oxidation process to remove NDMA and VOCs from contaminated water from wells E/F (Table 3), A (Table 4), or B (Table 4). For these studies, the water treatment apparatus included a plurality (i.e., 9) of ozone/advanced oxidation reactors arranged in series, adapted to allow sample of treatment water to be taken after each three reactors. Samples were identified as SP0, SP3, SP6, and SP9 to indicate how many reactors the water passed through prior to being tested for contaminant levels. SP0 samples passed through no ozone/advanced oxidation reactors, while SP3, SP6, and SP9 samples passed through 3, 6, or 9 reactors, respectfully. Since sample SP0 was treated with neither ozone nor hydrogen peroxide, it served as a control for measuring the affect of ozone and ozone/peroxide treatment of the various contaminants.

TABLE 3
Decontamination of water from well E/F using

| | Applied | Applied | Analytical Results (ppb) | | | | | |
|---|---|---|---|---|---|---|---|---|
| E/F | H₂O₂ | O₃ | | 1,1- | 1,2- | 1,2-DCE | | |
| Sample | (ppm) | (ppm) | NDMA | DCE | DCA | (cis/trans) | CHCl₃ | TCE |
| SP0 | 0 | 0.0 | 0.091 | 3.5 | 0.87 | 7.3 | 42 | 430 |
| SP3 | 2 | 5.0 | 0.039 | <0.5 | <0.5 | <0.5 | 30 | <0.5 |
| SP6 | 4 | 9.9 | 0.012 | <0.5 | <0.5 | <0.5 | 28 | <0.5 |
| SP9 | 5.9 | 15.1 | 0.004 | <0.5 | <0.5 | <0.5 | 26 | <0.5 |

TABLE 4
Decontamination of water from well B

| B | | | | | | | |
|---|---|---|---|---|---|---|---|
| | Applied | Applied | Analytical Results (ppb) | | | | |
| | H₂O₂ | O₃ | | 1,1- | 1,2- | Freon | |
| Sample | (ppm) | (ppm) | NDMA | DCE | DCA | 113 | TCE |
| SP0 | 0 | 0.0 | 13 | 3.1 | 1.1 | 0.56 | 20 |
| SP3 | 1.2 | 3.7 | 7.2 | <0.5 | <0.5 | 2.1 | <0.5 |
| SP6 | 2.4 | 7.5 | 3.1 | <0.5 | <0.5 | 0.74 | <0.5 |
| SP9 | 3.7 | 11.2 | 1.8 | <0.5 | <0.5 | <0.5 | <0.5 |

TABLE 5
Decontamination of water from well A

| | Applied | Applied | Analytical Results (ppb) | | | | | | |
|---|---|---|---|---|---|---|---|---|---|
| A | H₂O₂ | O₃ | | 1,1- | 1,1- | 1,2-DCE | Freon | | |
| Sample | (ppm) | (ppm) | NDMA | DCE | DCA | (cis/trans) | 113 | CHCl₃ | TCE |
| SP0 | 0 | 0.0 | 7.9 | 50 | 2.2 | 1.2 | 120 | 0.97 | 28 |
| SP3 | 1.2 | 3.1 | 4.7 | <0.5 | 1.1 | <0.5 | 33 | 0.63 | <0.5 |
| SP6 | 2.4 | 6.2 | 2.2 | <0.5 | 0.74 | <0.5 | 32 | 0.57 | <0.5 |
| SP9 | 3.7 | 9.4 | 0.94 | <0.5 | <0.5 | <0.5 | 22 | <0.5 | <0.5 |

As shown in Tables 3-5, even low doses of ozone and hydrogen peroxide were effective in removing VOCs such as 1,1-DCE, 1,2-DCA, 1,2-DCE (cis/trans), and TCE, which were generally present at <0.5 ppb using even the lowest doses of ozone and hydrogen peroxide. Freon 113 and chloroform (CHCI₃) were more refractory to destruction but their levels were reduced substantially using the higher doses of ozone and hydrogen peroxide, in some instances to <0.5 ppb.

The initial levels of NDMA varied considerably in water from different wells. The initial level in water from well E/F was only 0.091 ppb and could be reduced to 0.004 ppb using the higher doses of ozone and hydrogen peroxide. The initial level in water from wells B and A were 7.9 and 13 ppb, respectively, and reduced using the higher doses of ozone and hydrogen peroxide to 1.8 and <1 ppb, respectively.

These results demonstrated that advanced oxidation destroyed NDMA and various sentinel VOCs, in a dose-dependent manner, at reasonable levels of ozone and hydrogen peroxide. Ozone or ozone peroxide destroy NDMA permanently, and reformation is not an issue as in the case of UV photolysis. Ozone or ozone peroxide are expected to destroy DMA as readily as NDMA, and can therefore be used to remove DMA present in water, including DMA resulting from UV photolysis of NDMA.

Based, in part, on such data, a cost comparison of water treatment using UV energy and hydrogen peroxide, advanced oxidation, and UV energy in combination with advanced oxidation, demonstrated that UV treatment followed by advanced oxidation treatment was the least expensive method, have a periodic cost almost half that of UV and hydrogen peroxide treatment.

### V. Exemplary Apparatus

The present apparatus and methods can be performed using conventional UV apparatus having its effluent water directed to a suitable ozone or advanced oxidation apparatus. The ozone or advanced oxidation apparatus may be of the direct injection type or the sidestream type. UV treatment and ozone or advanced oxidation can also be combined in the same apparatus. Thus, the present apparatus and methods are not limited to any particular apparatus or operating configurations.

a. Description of the Apparatus

An exemplary apparatus for performing ozone or advanced oxidation treatment following UV treatment is a modular-design, plug-flow water treatment apparatus that features sidestream injection of ozone, and optionally hydrogen peroxide, combined with aggressive static mixing to maximize the dissolution of oxidants in influent water. Introducing ozone and hydrogen peroxide via sidestream mixing ensures maximum dissolution and dispersion of the agents in the main reactor, results in high mass transfer efficiency, allowing the use of lower doses of oxidants without the need for prolonged residence time in the main reactor or recycling influent water through the main reactor. The apparatus is ideal for low dose, high flow rate, single-pass water treatment applications, such as the treatment of water for use as potable water (including drinking water), for use in irrigation, industrial applications, toilet flushing, and the like, or for discharge to the environment (e.g., lakes, streams, or other bodies of water). The apparatus is also ideal for use downstream of UV treatment, for example, to destroy DMA produced by UV photolysis and to destroy a variety of other VOCs.

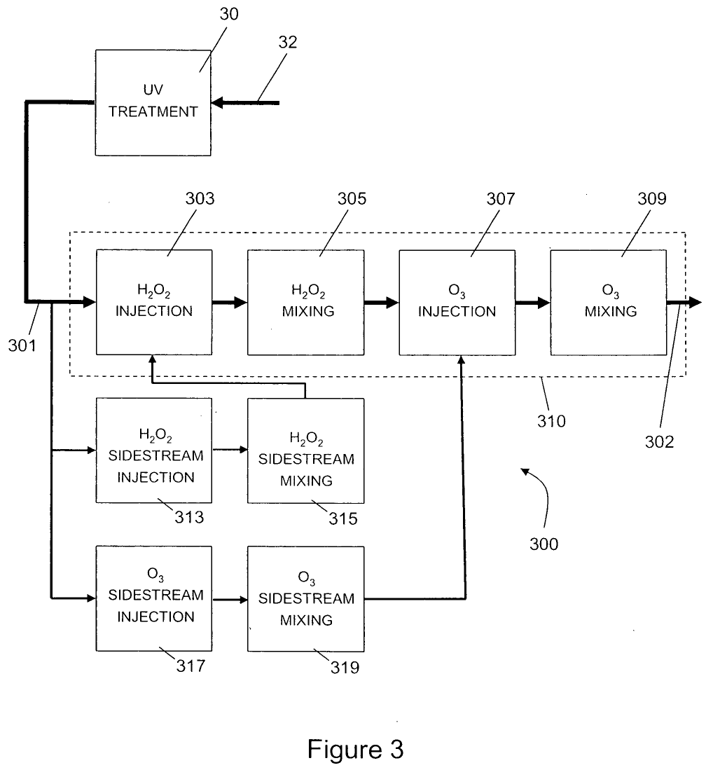

A schematic diagram of an exemplary modular ozone/advanced oxidation apparatus 300 coupled to a UV treatment apparatus 30 is shown in FIG. 3. Contaminated water enters an influent port 32 of a UV treatment apparatus 30, where photolysis occurs. Effluent water from the UV treatment apparatus 30 enters the influent port 301 of the main reactor 310 and treated water exits the effluent port 302. A portion of the influent water is directed away from the main reactor 310 for use in the ozone sidestream, which includes an ozone injector 317 upstream of a static mixer 319. Ozone injected via the sidestream may contact a further static mixer 309 to improve ozone dissolution in influent water in the main reactor 310. The ozone may be introduced under positive pressure with respect to the water in the sidestream or educted into the side stream, e.g., by venture effect.

In some configurations, a second portion of the of the influent water is directed away from the main reactor 310 for use in the hydrogen peroxide sidestream, which includes a hydrogen peroxide injector 313 upstream of a static mixer 315. Whereas hydrogen peroxide is conventionally added directly to the main reactor, or to influent water in a non-pressurized premixing chamber, a feature of the present apparatus is sidestream injection of hydrogen peroxide using an injector 313 coupled to a static mixer 315 to thoroughly mix hydrogen peroxide in the sidestream to improve hydrogen peroxide dissolution in the main reactor 310. More efficient hydrogen peroxide dissolution in the main reactor maximizes the production of hydroxyl radicals upon contact with ozone.

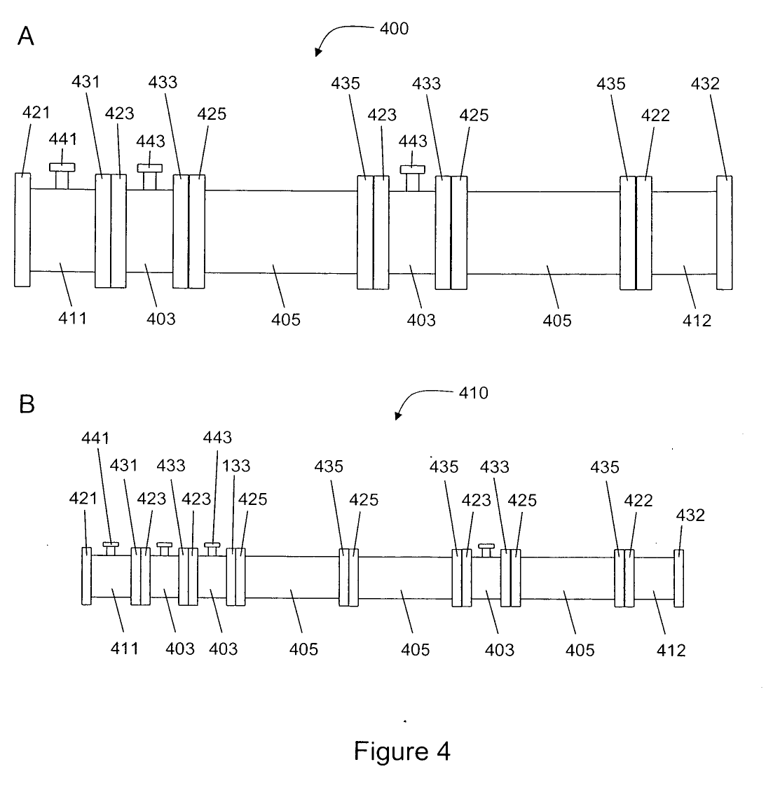

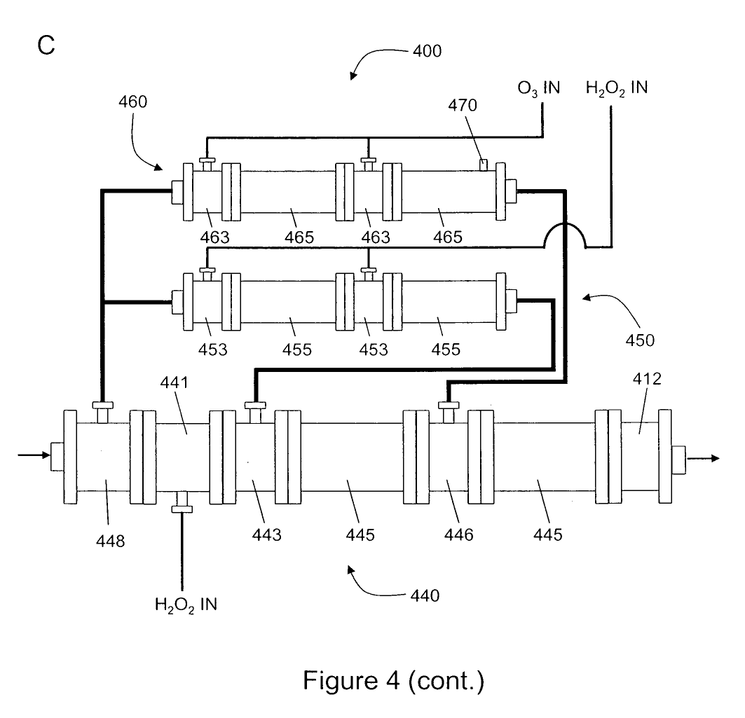

As illustrated in FIGS. 4A and 4B, the injector modules 403 and mixer modules 405 in the main reactor 400 (FIG. 4A) or sidestream 410 (FIG. 4B) are modular, allowing them to be combined in a variety of flow configurations and arrangement to optimize and customize the apparatus for a particular application. The modules for the sidestream 410 are typically of smaller diameter than those for the main reactor 400 but otherwise share modular construction features. Thus many of the following descriptions relate to both the main reactor and the one or more sidestreams, which are part of the apparatus.

The components of the apparatus are modular in form and function, allowing the components to be manufactured in quantity and assembled in different arrangements for use in custom applications. The major components of an apparatus are the injector modules and mixer modules in the main reactor and sidestream, which are similar in features although different in diameter, with the sidestream modules being substantially smaller in diameter than the main reactor modules.

Preferably, the fittings provided at the first ends 423 of each injector module 403 are the same (i.e., interchangeable) and the fittings provided at the second ends 433 of each injector module 403 are the same; and the fittings provided at the first ends 425 of each mixer module 405 are the same and the fittings provided at the second ends 435 of each mixer module 405 are the same (as above), such that arrangements of alternating injector modules and mixer modules can be assembled from the interconnecting modules of the same pipe or tubing diameter.

The first 423 and second 433 ends of each injector 403 and/or the first 425 and second 435 ends of each mixer 405 may be the same, allowing the injector modules 403 and mixer modules 405 to be operated in either direction, for ease of assembly. In other embodiments, the injector modules 403 and mixer modules 405 are designed to operate in a single flow direction, and the first 423, 433 and second 425, 435 ends may be the same or different. In yet further embodiments, the first 423, 425 and second 433, 435 ends of each injector module 403 and mixer module 405 are the same, allowing the assembly of sequential injectors 403 and/or sequential mixers 405, as in FIG. 4B.

Flange-type (i.e., flanged) module housings may be used, and may include a seal, such as an O-ring, to minimize leakage. Threaded fittings may also be used. In one example, injector modules are provided with male threads while mixing modules are provided with female threads to allow assembly injector modules with mixer modules. In another example, injector modules are provided with female threads while mixing modules are provided with male threads. In yet other examples, each first end of the injector modules and mixer modules are provided with a male (or female) thread and each second end of the injector modules and mixer modules are provided with a female (or male) thread, such that any number of injector modules and mixer modules can be assembled into an apparatus. Alternatively, all threads on modules can be male or female and couplers or unions can be used to connect the modules. Other tubing or pipe fittings may be used as required to connect to the effluent port of one or more suitable UV treatment apparatus.

An optional pre-mix module 411 may be added to introduce oxidants or other water treatment agents to the UV treated effluent water prior to contact with the reactor (FIGS. 4A and 4B). Instead or in addition to the pre-mix module, an optional post-mix contactor module 412 (or “contactor”) may be added to increase the residency time of the water in the presence of the oxidants. The contactor will typically have a larger internal volume than the injection and mixer modules, and is not shown to scale. Where present, the pre-mix module and contactor may include first 421, 422 and second 431, 432 ends, respectively. The first end 421 of the pre-mix module 411 and second end 432 of the contactor module 412, make be in the form of end caps that include fittings for attaching to influent and effluent water hookups, respectively.

A complete apparatus 400 for operation in an advanced oxidation mode is shown in FIG. 4C. The apparatus 400 includes a main reactor 440, an ozone sidestream 460 and a hydrogen peroxide sidestream 450. The main reactor 440 includes a diverter module 448 for diverting a portion of the effluent water from a UV treatment apparatus to the sidestreams 450, 460. The diverter module 448 or accompanying hookups may include one or more valves for modulating the amount of water diverted to each of the sidestreams (not shown), an optional pre-mix module 441 for injecting hydrogen peroxide, a hydrogen peroxide injector module 446 for injecting sidestream hydrogen peroxide, an ozone injector module 443 for injecting sidestream ozone, and mixer modules 445. The diverter module may be a component of the apparatus or a peripheral component.

Where hydrogen peroxide is introduced to a pre-mix module 441, it may be introduced by direct injection (i.e., without the use of a sidestream, as illustrated) or via an additional sidestream to maximize mixing and dissolution (not shown). The ozone sidestream 460 includes ozone injector modules 463 and mixer modules 465. The hydrogen peroxide sidestream 450 includes hydrogen peroxide injector modules 453 and mixer modules 455.

All or part of the water that flows through the hydrogen peroxide sidestream may be used in the ozone sidestream, in which case the apparatus, system, and method may involve the consecutive (i.e., sequential) addition of hydrogen peroxide, and then ozone, in a sidestream configuration, prior to introduction of the oxidants to a main reactor. Introduction of concentrated hydrogen peroxide, or hydrogen peroxide mixed in a sidestream, to the ozone sidestream, prior to introduction to the main reactor, is effective in controlling bromate formation.

In some cases, the water used in the ozone and/or hydrogen peroxide sidestream is from the same source as the water into which the sidestream water and oxidants are mixed (e.g., effluent water from a UV treatment apparatus). In this manner, water destined for a sidestream is diverted from the water flowing to the main reactor. Alternatively, the sidestream water originates from a different source than the water that enters the main reactor, which may be water that has not been UV treated. In such cases, the sidestream water may include higher or lower levels of contaminants compared to the water supplied to the main reactor.

The apparatus may further include one or more degassing ports to release residual gas pressure resulting from the presence of undissolved ozone, oxygen, or air (depending on the particular oxidant gasses used). Degassing (or “off-gassing”) ports may be provided in the main reactor, preferably downstream of the site of injection one or more gases, to allow the release of gas pressure produced by undissolved oxidant gases. Alternatively or additionally, degassing ports may be provided in a sidestream (typically the gas oxidant sidestream) to allow the release of gas pressure prior to mixing in the main reactor. Degassing ports may be provided in an additional module that can be assembled in combination with injector and mixer modules (i.e, a discrete “degassing module”) or combined with an existing module, such as a mixer module. FIG. 4C illustrates a degassing port 270 located in mixer module 265, although other locations of one or more degassing ports are contemplated.

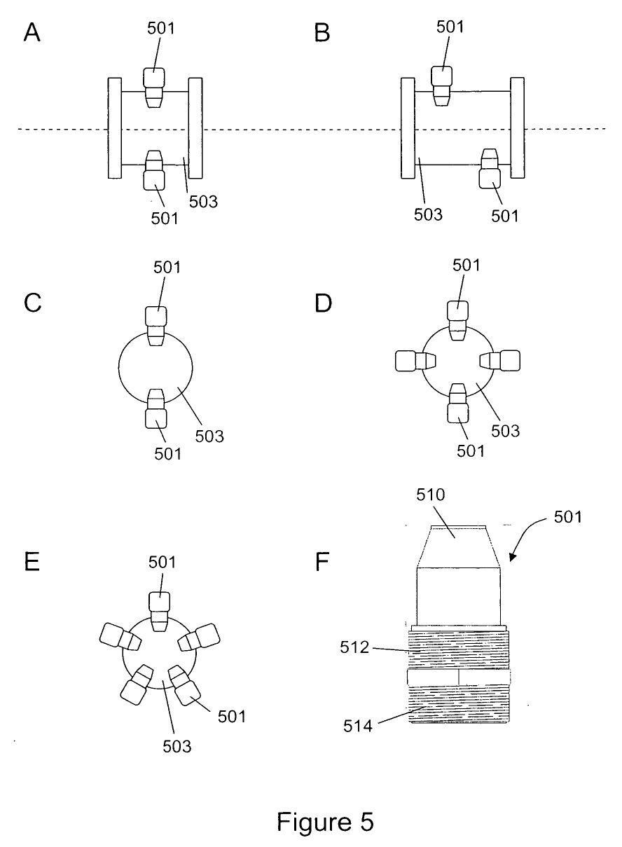

FIGS. 5A-5F illustrate several different arrangements of the injectors 501 within an injection module 503. One or more injectors 501 may be present in each injection module 503. Where a plurality of injectors are present, the injectors 501 may be arranged in the same plane (FIG. 5A) or through several planes (FIG. 5B) perpendicular to the major axis (dotted line) of an injector module 503. A plurality of injectors 501 may be arranged in opposed pairs (FIGS. 5C and 5D) or radially (i.e., in a star-burst pattern; FIG. 5E) in the injector module 503, so as to maximize the distribution of the injected oxidant in the reactor. To simplify the drawings, only the inside wall of the injector module 503 (and not the flange) is shown in FIGS. 5C-5E.

An exemplary injector is illustrated in FIG. 5F. The injector is of a conventional nozzle-design, and includes a nozzle portion 510, threads for engaging a tapped hole on an injector module 512, and threads for connecting to an ozone or hydrogen peroxide supply 514 (which may be from a sidestream). The size and number of injectors 501 are selected for the application and the nozzles may be designed to produce a fan or cone-shaped spray, further distributing the injected oxidant.

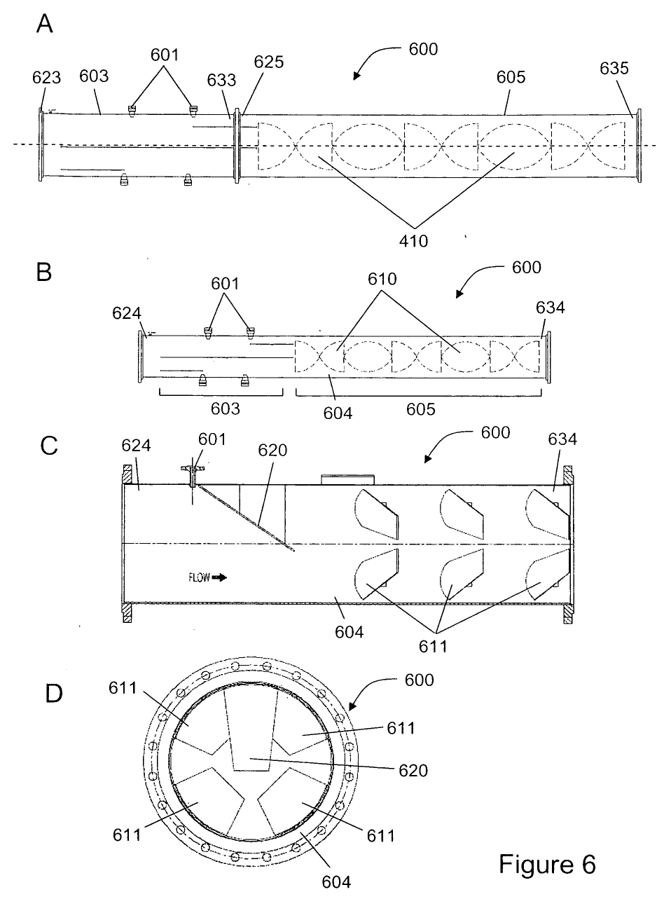

FIGS. 6A-6D illustrate exemplary apparatus including different static mixer 605 configurations. FIG. 6A shows a side view of an apparatus 600 having a separate injector module 603 and mixer module 605. The first 623 and second 633 ends of the injector module 603 and first 625 and second 635 ends of the mixer module 605 are indicated. The exemplary injector module 603 includes four injectors 601, each in a separate plane normal to the axis (dashed line) of the injector 603.

The apparatus 600 shown in FIG. 6A includes a mixer module 605 includes a vane-type static mixer 610 comprising a substantially flat twisted member that induces a vortex in water passing through the mixer 605. Such mixers are exemplified by the Chemineer model WVM mixer (Chemineer, Derby, U.K.). The apparatus 600 shown in FIG. 6B include the feature that the mixer module 604 shares a common chamber with the injector module, thereby forming a combined injector-mixer module 604 that includes an injector module 603 portion and a mixer module 605 portion. Such combined modules may be used in many embodiments of the apparatus, particularly where it is desirable to provide a “core” apparatus, having a basic level of capacity and performance, which can be modified as needed using additional modules to achieve a preselected level of capacity or performance. Additional or “add-on” injector modules and mixer modules may also be in the form of combined injector-mixer modules.

A mixer module 605 or a combined injector-mixer module 604 may alternatively include a tab-type mixer, in which one or more tabs 611 extending from the interior wall of the mixing module 604 causes turbulence or vortex motion in water passing through the mixing module (FIG. 6C). An additional pre-distribution tab or deflector 620 may be located downstream of the injector 601 to direct the injected oxidant to the center of the static mixer. The cross-sectional view (FIG. 6D) of the apparatus illustrates the mixing tabs 611 and the additional tab or deflector 620 downstream of the injector 601. Tab mixers are exemplified by the Chemineer model KMS mixer (Chemineer, Derby, U.K.).

The distance between any one or more injectors 601 and the mixer module 605 (or the components of the mixer module 605) is generally not critical but may be selected to maximize the mixing of oxidants with influent water upon introduction into the sidestream. A plurality of injector modules, for injecting the same or different oxidants directly or via sidestream, can be arranged in series upstream of a mixer module. Multiple mixer modules, of the same or different design, can be arranged in series to improve mixing. A goal of the present apparatus, systems, and methods is to maximize mixing efficiency while minimizing energy consumption; therefore, it is generally preferred to use the minimum number of efficient mixers required to achieve thorough mixing and oxidant utilization, which may be reflected by the gas to liquid ration, described, herein.

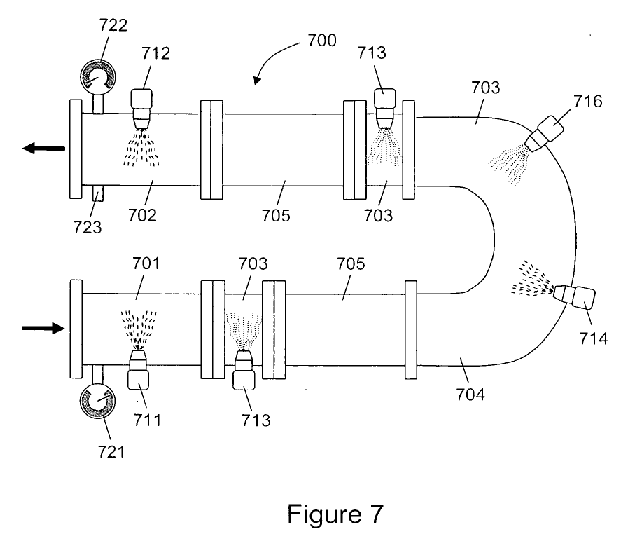

FIG. 7 illustrates an exemplary main reactor 700 comprising a pre-mix module 701, two injector modules 703, two mixer modules 705, and a post-mix module 702. The injector modules 703 include ozone injectors 713 for injecting ozone provided via a sidestream. The pre-mix module 701 and post-mix module 702 include hydrogen peroxide injectors 711, 712 for injecting hydrogen peroxide provided via a sidestream. The sidestream ozone and/or hydrogen peroxide modules may also be connected by elbows, thereby making the overall apparatus more compact. In this case, an elbow 704 is use to connect a mixer module 705 to an injector module 703. The elbow 704 may further include a sidestream or direct hydrogen peroxide injector 714 and/or a sidestream or direct ozone injector 716.

The apparatus in FIG. 7 also includes an optional influent water pressure gauge 721, effluent water pressure gauge 722, and effluent water sample port 723, which can be used to monitor the performance of the apparatus and inform adjustments and modifications.

b. Operating Configurations

The modular nature of the exemplary apparatus allows the assembly of specialized, on-site treatment units, with a minimum of components of minimum complexity. Modules are readily added or subtracted to the apparatus to optimize efficiency and performance. In this manner, an apparatus may be installed in an existing UV treatment facility, setup with an initial number of modules, tested for efficiency and performance, and modified with additional injector modules and mixer modules (or combined modules) until satisfactory effluent water quantity and quality are obtained. This avoids the high cost and risk of designing a specialized apparatus for each application, in some cases with limited scalability and ability to substitute components.

The apparatus can be operated in an ozone dissolution mode, in which ozone is injected into the reactor via a sidestream without the addition of hydrogen peroxide, or in advanced oxidation mode, in which ozone is injected into the reactor via a sidestream and hydrogen peroxide is injected either directly into the main reactor or via a sidestream into the main reactor

Preferred apparatus for operation in an ozone dissolution mode include a main reactor and at least one sidestream for injecting and mixing ozone prior to its introduction to the main reactor. The sidestream includes an injector module and a mixer module, which may be discrete components or combined in a common housing. The main reactor may include any number of ozone injectors and mixers in addition to those located in the sidestream. In some embodiments, a mixer module is provided in the main reactor immediately downstream of the sidestream ozone injection port, such that sidestream-injected ozone is maximally distributed in the main reactor. The mixer module may be of a vane-type, a tab-type, other mixer types known in the art, or a combination thereof. Where a plurality of mixers is used, it may include one or more of each type.

Apparatus for operation in an advanced oxidation mode further include at least one hydrogen peroxide injector for injecting and mixing hydrogen peroxide in the main reactor. Hydrogen peroxide may be introduced directly or via a sidestream to maximize mass transfer efficiency and reduce the amount of hydrogen peroxide required to reduce the levels of contamination in influent water by a preselected amount. As above, the sidestream includes an injector module and a mixer module, which may be discrete components or combined in a common housing. The main reactor may include any number of hydrogen peroxide injectors and mixers in addition to those located in the sidestream. In one example, a mixer module is provided in the main reactor immediately downstream of the sidestream hydrogen peroxide injection port, such that sidestream-injected hydrogen peroxide is maximally distributed in the main reactor.

A plurality of injector modules may be positioned in series, followed by one or more mixer modules. A single injector module may include injectors for ozone, hydrogen peroxide, or both. For example, where a plurality of injectors are located in the same injector module, some (i.e., a first portion) of the injector may be for injecting ozone, while other (i.e., a second portion) are for injecting hydrogen peroxide. Alternatively, “ozone only” injector modules and “hydrogen peroxide-only” injector modules may be used, each having injectors only for the indicated oxidant. Ozone injector modules and hydrogen peroxide injection modules may be arranged in an alternating manner or in another logical manner to deliver the oxidants for maximum efficiency and performance.

While ozone and hydrogen peroxide can be injected in any order, it may be preferably to first inject hydrogen peroxide upstream of ozone, giving the hydrogen peroxide time to thoroughly mix with effluent water from a UV treatment apparatus prior to exposure to ozone. Addition of hydrogen peroxide at an early stage reduces the reformation of NDMA and reduces the formation of bromate, which occurs in the present of elevated ozone concentrations but is reduced in the presence of hydrogen peroxide (see, e.g., U.S. Pat. Nos. 5,851,407 and 6,024,882). Where maximum hydroxyl radical formation and reduced consumption of hydrogen peroxide are desired, ozone may be injected first to react with organic compounds present in the influent water to produce hydroxyl radicals or other radicals, followed by the addition hydrogen peroxide to react with residual ozone to produce additional hydroxyl radicals.

Another way to control bromate formation is to introduce concentrated hydrogen peroxide, or a portion of the diluted hydrogen peroxide from a hydrogen peroxide sidestream, into the ozone sidestream, thereby controlling bromate formation in the ozone sidestream.

Mixer modules may be of a vane-type or a tab-type, or both. Where a plurality of mixer modules are used, they may include one or more of each type, thereby exploiting the advantages of each type of mixer. A mixer module may be positioned after each ozone and/or hydrogen peroxide injector module. Alternatively, a mixer module may be positioned after each ozone injector module-hydrogen peroxide injector module pair. A mixer module may also be positioned after a plurality of ozone injector modules and/or hydrogen peroxide injector modules.

A pre-mix module may be included upstream of the main reactor, and may include ozone and/or hydrogen peroxide injectors. Pre-mix modules may include inlet ports (which may be injectors) for adding hydrogen peroxide to influent water prior to ozone injection. Ozone only operation may also benefit from the use of a contactor located downstream of the main reactor for allowing the ozone sufficient time to dissipate in the water and affect decontamination. Such downstream ozone contactors may take the form of a pipeline, baffled or unbaffled tank (including an “over” or “under” baffled tank), or similar devices. Downstream ozone contactors may be integrated into the main reactor or maybe a stand-alone component downstream of the main reactor. Contactor modules may include inlet ports (which may be injectors) for adding hydrogen peroxide to effluent water following ozone injection, e.g., to control bromate formation. Where the apparatus is operated in an ozone dilution mode, without hydrogen peroxide, the pre-mix and/or contactor modules may be used to deliver additional ozone.

A downstream gas-liquid separator may be included to release excess ozone gas, oxygen, and or air, e.g., to reduce corrosion in downstream equipment and to reduce fugitive emissions to promote health and safety. Excess gas may be disengaged (vented) using either a pipe or a vessel of larger diameter than the reactor, such that the velocity of the water is reduced, increased surface area is exposed, and excess gas is allowed to vent through a vent valve and, optionally, an ozone destruct unit. In some cases, the larger diameter pipe or vessel may be baffled or the liquid stream containing excess gas is introduced into the larger pipe or vessel on a tangent to induce a vortex to assist in evolving excess gas.

The initial main reactor and sidestream flow rates can be preselected for a particular application and the optimized following installation and initial testing. Guidelines for the initial set up of an apparatus are provided in Tables 8 and 9, below; however these are only examples.

TABLE 8
Ozone and oxygen flow parameters.

| | | | | | | | | | |
|---|---|---|---|---|---|---|---|---|---|
| Flow rate (MGD) | 1.0 | 1.0 | 1.0 | 2.5 | 2.5 | 2.5 | 5.0 | 5.0 | 5.0 |
| O₃ dose (mg/L) | 1.0 | 5.0 | 15.0 | 1.0 | 5.0 | 15.0 | 1.0 | 5.0 | 15.0 |
| O₂ flow (slpm) | 0.7 | 3.4 | 10.1 | 45.1 | 225.4 | 676.3 | 90.2 | 450.9 | 1352.6 |
| SS flow rate (%) | 7.5 | 15 | 25 | 7.5 | 15 | 25 | 7.5 | 15 | 25 |
| SS flow rate (GPM) | 52.1 | 104.2 | 173.6 | 130.2 | 260.4 | 434.0 | 260.4 | 520.8 | 868.1 |
| G/L ratio (total) | 0.007 | 0.036 | 0.108 | 0.007 | 0.036 | 0.108 | 0.007 | 0.036 | 0.108 |

SS = Sidestream;
GPM = gallons per day;
MGD = million gallons per day

TABLE 9
Hydrogen peroxide flow parameters.

| | | | | | | |
|---|---|---|---|---|---|---|
| Flow rate (MGD) | 1.0 | 1.0 | 2.5 | 2.5 | 5.0 | 5.0 |
| H₂O₂ dose (mg/L) | 0.5 | 7.5 | 0.5 | 7.5 | 0.5 | 7.5 |
| H₂O₂ flow rate (ml/min) | 3.3 | 49.7 | 8.3 | 124.3 | 16.6 | 248.6 |
| Sidestream flow rate (%) | 2.5 | 5.0 | 2.5 | 5.0 | 2.5 | 5.0 |
| Sidestream flow rate (GPM) | 17.4 | 35.0 | 43.4 | 86.8 | 86.8 | 173.6 |
| 0.75″ (feet/sec) | 9.1 | — | — | — | — | — |
| 1″ (feet/sec) | 5.9 | 11.7 | 14.7 | — | — | — |
| 1.5″ (feet/sec) | 2.5 | 5.0 | 6.3 | 12.5 | 12.5 | — |
| 2″ (feet/sec) | — | — | — | — | 7.6 | 15.2 |
| 2.5″ (feet/sec) | — | — | — | — | 5.1 | 10.2 |

SS = Sidestream;
GPM = gallons per day;
MGD = million gallons per day

An important consideration in designing the ozone sidestream is the gas/liquid (G/L) ratio, which reflects the amount of ozone gas dissolved in the influent water. A high gas/liquid ratio suggests that ozone gas is not efficiently dissolved in the influent water to affect oxidation of contaminants. A low gas/liquid ratio suggests that ozone gas is efficiently dissolved in the influent water where it is available to oxidize contaminants in the water. A high gas/liquid ratio can be reduced by providing additional mixing or increasing velocity through the ozone sidestream.

Generally, the higher the sidestream velocity, the better the mixing of injected oxidants in the sidestream water, and ultimately the main reactor. However, higher sidestream velocities are associated with increased energy costs, which reduce the overall efficiency and increase the environmental impact of the process. Combining sidestream injection of oxidants with efficient static mixers provides efficient mixing at lower flow rates and energy costs. Exemplary sidestream flow rates are from about 2 to about 20 feet per second (FPS), from about 3 to about 15 FPS, or even from about 5 to about 10 FPS, although flow rates outside these ranges may provide satisfactory results with suitable equipment.

In addition to being coupled to a UV treatment apparatus, the present modular, compact apparatus can be optimized or customized by adding additional modules. For example, the apparatus may be coupled with upstream or downsteam ultraviolet biofiltration processing operations (including but not limited to the use of membrane biofilm reactor (MBfR) processing), granulated activated charcoal (GAC) or powdered activated charcoal (PAC) processing operations, reverse osmosis (RO) processing operations, and/or chemical treatment processing operations.

The advantages of the present modular apparatus may be embodied in a kit of parts, or “kit” to be supplied to a municipality, company, or individual for adding ozone dissolution and/or advanced oxidation capabilities to UV treatment apparatus or facilities. Such kits may include one or more main reactor injector modules and mixer modules, one or more sidestream injector modules and mixer modules, a plurality of injectors, connectors and fittings, and instructions for installing and using the apparatus. Kits may be sized for particular application as determined by throughput capacity, influent water contaminant levels and types, effluent water contaminant requirements, existing equipment and fittings, and other considerations.

Kits may include several injector modules with the same or different injector configurations. The injector modules may be adapted to accept a plurality of injectors, such as by including threaded openings for injectors or threaded plugs to seal unused openings. The kits may also include several mixer modules with the same or different injector configurations. Where desired, additional injector modules and mixer modules can be combined with the components of the kit.

Kits may be packaged for treating a preselected volume of water or level of contamination, and may be accompanied by written or electronic instructions, speadsheets, and other documents or software relating to the installation, start-up, and optimization of the apparatus. Such kits can be installed and operated by a customer or installed by specially trained personnel and operated by a customer.

c. Selection of Oxidants

Variations of the ozone dissolution process utilize ozone, oxygen, air (which includes oxygen), ozone and oxygen, ozone and air, oxygen and air, or ozone, oxygen and air, as gas oxidants. Such combinations of ozone, oxygen, and/or air can be used in an advanced oxidation process/mode along with hydrogen peroxide. While the present apparatus and methods are mainly described with respect to the use of ozone (with or without hydrogen peroxide), ozone, oxygen, and/or air can in some cases be used in place of ozone in some embodiments. The selection of the particular gas oxidant(s) largely depends on the types and levels of contaminants present in the influent water, the additional decontamination process operations that are used in combination with the present apparatus, systems, and methods, and the proposed use of the decontaminated water.

When operating the apparatus in an advanced oxidation mode, an excess of hydrogen peroxide may be used where bromate formation is an issue. In particular, bromate formation can be reduced using a plurality of hydrogen peroxide sidestream injectors to maintain a high level of hydrogen peroxide in the main reactor. Bromate formation can also be controlled via pH adjustment and/or the addition or chlorine or ammonia. Any of the present apparatus, systems, and methods can be adapted to permit the introduction of such bromate formation-controlling agents. Conversely, an excess of ozone, or both ozone and hydrogen peroxide, may be used to ensure that discharged (treated) water includes residual oxidants to promote further decontamination, even downstream of the present apparatus.

d. Residence Times

A feature of the present apparatus and methods is the ability to greatly reduce the residence time of contaminated water in a reactor that is required for the substantial removal of a particular contaminant, such as DMA and VOCs. Residence time refers to amount of time a given volume of contaminated water must spend in a reactor (or series of reactors) to achieve a preselected amount of reduction of a contaminant following UV treatment. Unless otherwise specified, residence time includes time spent in a downstream contactor, if such a component is present, but not time spent in a UV treatment apparatus. Residence time in the main reactor can be modulated by controlling the flow rate/velocity of influent water through the main reactor.

Convention ozone and ozone/peroxide water treatments require several minutes of residence time (e.g., 8 or more minutes) to provide adequate reduction of contaminant. In contrast, the efficiency of the modular apparatus, system, and methods permits adequate contaminant removal in minimal residency time, typically only a few minutes, if not seconds. Exemplary residency times are about 10 seconds to about five minutes, for example, 10, 15, 20, 25, 30, 40, or 50 seconds, or 1, 1.5, 2, 2.5, 3, 3.5, 4, 4.5, or 5 minutes. This reduction in residence time increases the throughput of contaminated water, allowed a greater volume of water to be treated and reused.

Other aspects of the present apparatus and method will be apparent to one skilled in the art based on the foregoing disclosure and appended claims. Various modifications and variations can be made without departing from the spirit or scope of the subject matter described herein.

What is claimed is:

1. A method for removing N-nitroso-dimethylamine (NDMA) and NDMA derivatives from water, comprising contacting the water with UV light, and then contacting the water with ozone, thereby reducing the amount of NDMA and NDMA derivatives.

2. The method of claim 1, wherein the NDMA derivative is dimethylamine (DMA).

3. The method of claim 1, wherein the ozone is provided in the absence of hydrogen peroxide.

4. The method of claim 1, wherein the ozone is provided in combination with hydrogen peroxide.

5. The method of claim 1, wherein the water further included volatile organic compounds (VOCs) and the method reduces the amount of VOCs in the water.

6. The method of claim 1, wherein the method disinfects the water.

7. The method of claim 6, wherein the disinfecting includes killing viruses and coliforms.

8. The method of claim 1, performed in a modular sidestream injection apparatus.

9. A method for removing N-nitroso-dimethylamine (NDMA) and NDMA derivatives from water, comprising contacting the water with ozone, and then contacting the water with UV light, thereby reducing the amount of NDMA and NDMA derivatives.

10. The method of claim 9, wherein the NDMA derivative is dimethylamine (DMA).

11. The method of claim 9, wherein the water further included volatile organic compounds (VOCs) and the method reduces the amount of VOCs in the water.

12. The method of claim 9, wherein the ozone is provided in the absence of hydrogen peroxide.

13. The method of claim 9, wherein the ozone is provided in combination with hydrogen peroxide.

14. The method of claim 9, wherein the method disinfects the water.

15. The method of claim 14, wherein the disinfecting includes killing viruses and coliforms.

16. The method of claim 9, further comprising contacting the water with ozone after contacting the water with UV light.

17. The method of claim 9, wherein the UV light causes the formation of hydroxyl radicals from ozone.

18. The method of claim 9, wherein treatment of the water with ozone increases the UV transmittance of the water prior to treating the water with UV light.
